SEQUENCE LISTING (14 sequences)

SEQ ID NO: 1 - DNA, 19 bp, Artificial Sequence
  Description of Artificial Sequence Synthetic primer
tctagaaaaacctgccaaa

SEQ ID NO: 2 - DNA, 19 bp, Artificial Sequence
  Description of Artificial Sequence Synthetic primer
taccaggaaatgagcttga

SEQ ID NO: 3 - DNA, 20 bp, Artificial Sequence
  Description of Artificial Sequence Synthetic primer
accagagatattgtggatgg

SEQ ID NO: 4 - DNA, 21 bp, Artificial Sequence
  Description of Artificial Sequence Synthetic primer
ttgagatttttggggtctatg

SEQ ID NO: 5 - DNA, 20 bp, Artificial Sequence
  Description of Artificial Sequence Synthetic primer
tccagatttggacctaaaag

SEQ ID NO: 6 - DNA, 20 bp, Artificial Sequence
  Description of Artificial Sequence Synthetic primer
ctctgaagagcgaaaaggta

SEQ ID NO: 7 - DNA, 20 bp, Artificial Sequence
  Description of Artificial Sequence Synthetic primer
tgaggaggaacaagaagatg

SEQ ID NO: 8 - DNA, 20 bp, Artificial Sequence
  Description of Artificial Sequence Synthetic primer
tgaggaggaacaagaagatg

SEQ ID NO: 9 - DNA, 20 bp, Artificial Sequence
  Description of Artificial Sequence Synthetic primer
cagttggaactcttgagtgg

SEQ ID NO: 10 - DNA, 20 bp, Artificial Sequence
  Description of Artificial Sequence Synthetic primer
atataaccatccccaaggtc

SEQ ID NO: 11 - DNA, 20 bp, Artificial Sequence
  Description of Artificial Sequence Synthetic primer
ggaagacattgaacgtatgg

SEQ ID NO: 12 - DNA, 20 bp, Artificial Sequence
  Description of Artificial Sequence Synthetic primer
aatcaacctcttcaatggtg

SEQ ID NO: 13 - DNA, 20 bp, Artificial Sequence
  Description of Artificial Sequence Synthetic primer
gctctggaaaggatgatatg

SEQ ID NO: 14 - DNA, 20 bp, Artificial Sequence
  Description of Artificial Sequence Synthetic primer
gatcccatattttcatccaa

(19) United States
(12) Patent Application Publication
(10) Pub. No.: US 2006/0234272 A1
(43) Pub. Date: Oct. 19, 2006

(54) Using gene panels to predict tissue sensitivity to ionizing radiation

(75) Inventors: Andrew J. Wyrobek, Walnut Creek, CA (US); Matthew A. Coleman, Oakland, CA (US); David O. Nelson, Oakland, CA (US)

Correspondence Address: Eddie E. Scott;Assistant Laboratory Counsel, Lawrence Livermore National Laboratory, P.O. Box 808, L-703, Livermore, CA, 94551, US

(73) Assignee: The Regents of the University of California

(21) Appl. No.: 11/390,584

(22) Filed: Mar. 27, 2006

Related U.S. Application Data
(60) Provisional application No. 60/667,345, filed on Mar. 31, 2005.

(51) Int. Cl. C12Q 1/68

(52) U.S. Cl. 435/6

(57) ABSTRACT

A method of predicting sensitivity to radiation comprises selecting at least one of a first panel of genes associated with increased chromosomal damage and a second panel of genes associated with reduced chromosomal damage. The tissue is exposed to radiation. The RNA of the tissue is measured providing measured RNA of the first panel of genes associated with increased chromosomal damage and providing measured RNA of the second panel of genes associated with reduced chromosomal damage. Sensitivity to radiation is predicted using at least one of the measured RNA of the first panel of genes associated with increased chromosomal damage and the measured RNA of the second panel of genes associated with reduced chromosomal damage.

### CROSS-REFERENCE TO RELATED APPLICATIONS

This application claims the benefit of U.S. Provisional Patent Application No. 60/667,345 filed Mar. 31, 2005 by Andrew J. Wyrobek, Matthew A. Coleman, and David O. Nelson titled “Gene panels that predict differential tissue sensitivities to radiation-induced toxicity.” U.S. Provisional Patent Application No. 60/667,345 filed Mar. 31, 2005 titled “Gene panels that predict differential tissue sensitivities to radiation-induced toxicity” is incorporated herein by this reference.

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to ionizing radiation and more particularly to predicting tissue sensitivity after cellular, tissue, and whole body exposures to ionizing radiation.

2. State of Technology

United States Patent Application No. 2003/0165956 by Craig, W. Stevens et al for an electrophoretic assay to predict risk of cancer and the efficacy and toxicity of cancer therapy, published Sep. 4, 2003, provides the following state of technology information: “Approximately 1.2 million Americans are expected to develop cancer this year, and one patient in three will receive radiotherapy during the course of their disease. Since radiation complications occur in 5-10% of these patients, this means that 20,000 to 40,000 patients will suffer long term complications per year. This problem will become more serious as cancer survival increases. Radiation complications are dependent on the organ irradiated, the volume of that organ irradiated, how the radiation is delivered (daily dose and total dose), and the intrinsic radiosensitivity of the patient. Complications are not manifest in all patients at high risk, or may be manifest quite late after treatment. Late radiation complications are often modeled as a stochastic process, but can be affected by DNA repair problems.”

The July/August 2003 article “Cells Respond Uniquely to Low-Dose Ionizing Radiation,” in the July/August 2003 issue of Science & Technology Review, provides the following state of technology information: “For decades, scientists have studied the cellular and genetic damage that follows exposure to high doses of ionizing radiation such as those resulting from nuclear accidents or cancer radiotherapy. Much less is known about cellular response to low doses of ionizing radiation—about 0.1 gray and below—such as that absorbed by our bodies during medical procedures and normal occupational exposures or while flying in an airplane.”

The August 2002 article “Adaptive response induction and variation in 3 human lymphoblastoid cell lines,” by Karen J. Sorensen, Cristina M. Attix, Allen T. Christian, Andrew J. Wyrobek, and James D. Tucker, in Mutation Research, Vol. 519, Issues 1-2, pp. 15-24, August 2002, provides the following state of technology information: “Low doses of ionizing radiation were first shown to modify the outcome of subsequent high doses of radiation to human cells in 1984 by Olivieri et al., who found that peripheral blood lymphocytes cultured in the presence of [3H]thymidine showed a reduced frequency of chromosome aberrations following X-ray exposure. This phenomenon, reviewed by Shadley and Wolff, became known as the adaptive response and is described as the ability of a low “priming” dose of radiation (usually less than 10 cGy) to modify the effects of a subsequent “challenge” dose (1-2 Gy). Adaptation has been observed in many different mammalian systems including various human and animal cell lines, mice, and rabbits.”

The October 2005 article “Low-Dose Irradiation Alters the Transcript Profiles of Human Lymphoblastoid Cells Including Genes Associated with Cytogenetic Radioadaptive Response,” by Matthew A. Coleman, Eric Yin, Leif E. Peterson, David Nelson, Karen Sorensen, James D. Tucker, and Andrew J. Wyrobek, in Radiation Research Vol. 164, No. 4, pp. 369-382, October 2005, provides the following state of technology information: “Exposure to low doses of ionizing radiation (<10 cGy) alters gene expression profiles in cells and animal tissues but, under certain circumstances, protects cells against the damaging effects of subsequent higher-dose exposures. This protective phenomenon, generally known as the adaptive response, has been broadly observed in mammalian systems and can reduce cytogenetic damage, enhance survival, increase resistance to infection, and reduce tumor incidence.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

There are currently no effective biomarkers of radiation sensitivity. The general consensus is that individual biomarkers will be insufficient and that panels of molecular biomarkers will be needed. Exposure to low-dose ionizing radiation (IR) (<10 cGy) alters gene-expression profiles in cells and animal tissues but, under certain circumstances, protects cells against the damaging effects of subsequent higher-dose exposures. This protective phenomenon, generally known as the adaptive response (AR), has been broadly observed in mammalian systems and can reduce cytogenetic damage, enhance survival, increase resistance to infection, and reduce tumor incidence.

It has been established that low-dose irradiation alters the expression of genes associated with diverse cellular functions and different forms of ionizing radiation show qualitative differences in the pathways affected (i.e., γ vs. ρ radiation). There have been several studies of gene-transcript expression in cells exposed to IR, yet only two have assessed the global cellular effects of low-doses (<10 cGy) and none have used gene transcript profiling to investigate the mechanisms of AR.

The AR phenotype has been associated with DNA damage repair and stress response functions based on functional and single-gene investigations. Exogenous endonucleases that generate DNA breaks have induced AR suggesting that DNA damage is involved, and inhibitors of protein synthesis can block AR, suggesting that AR requires de novo protein synthesis. Inhibitors of the DNA repair-related protein Poly-(ADP-ribose) polymerase (PARP) can block AR, further implicating repair processes. DNA-PK, ATM, and TP53, which are involved in DNA damage recognition and signaling, have also been implicated in AR. It has been suggested that TP53 plays a major role in AR via a p38MAPK signaling pathway along with other effectors that may include BRCA1, BRCA2, IRF-1, Rb, ERK1/2 and JNK/SAPK. The DNA repair protein DIR1 has been implicated in AR by increasing the rate of repair and APE1, a base excision repair endonuclease, may be involved in AR by linking repair to oxidative pathways. Although there have been numerous studies of individual genes and their proteins, there has been no genomic-scale assessment of the cellular responses of cells to low-dose radiation nor of the gene expression associations with the AR phenomenon.

The present invention provides a method of predicting sensitivity to radiation. The method comprises selecting at least one of (A) a first panel of genes associated with increased chromosomal damage and (B) a second panel of genes associated with reduced chromosomal damage. The tissue is exposed to radiation. The RNA of the tissue of the first panel of genes and the second panel of genes are measured. The measurements provide measured RNA of the first panel of genes associated with increased chromosomal damage and measured RNA of the second panel of genes associated with reduced chromosomal damage. Sensitivity to radiation is predicted using at least one of (A) the measured RNA of the first panel of genes associated with increased chromosomal damage and (B) the measured RNA of the second panel of genes associated with reduced chromosomal damage.

In one embodiment, the present invention provides a method of predicting tissue sensitivity to radiation comprising selecting a panel of genes associated with increased chromosomal damage, selecting a panel of genes associated with reduced chromosomal damage, exposing the tissue to radiation, measuring RNA of the tissue, and predicting sensitivity to radiation using the panel of genes associated with increased chromosomal damage and the panel of genes associated with reduced chromosomal damage.

In another embodiment, the present invention provides a method of using tissue for predicting sensitivity to radiation comprising selecting a first panel of genes associated with increased chromosomal damage, selecting a second panel of genes associated with reduced chromosomal damage, exposing the tissue to a priming dose of radiation, waiting for a time period and exposing the tissue to a challenge dose of radiation, waiting for a time period and measuring RNA of the tissue providing measured RNA of the first panel of genes associated with increased chromosomal damage and providing measured RNA of the second panel of genes associated with reduced chromosomal damage, and predicting sensitivity to radiation by comparing the measured RNA of the first panel of genes associated with increased chromosomal damage and the measured RNA of the second panel of genes associated with reduced chromosomal damage.

The present invention is based, in part, on the results of genome-scale gene expression studies in human cells exposed to ionizing radiation using gene-transcript microarrays. Three hypotheses were tested in Applicants' study: (a) exposure of cells to acute low-dose irradiation (priming dose) prior to an acute high-dose exposure (challenge dose) induces changes in the transcriptome profiles that persist beyond the challenge dose, (b) specific gene-transcript changes induced by the priming dose are independent of whether a cell line will show AR or not, while (c) transcript changes in other genes will be predictive of AR outcomes. Applicants previously characterized numerous human lymphoblastoid cell lines (LCL) for cytogenetic AR phenotypes by micronucleus analyses, and selected three lines for the current study that were reproducibly adapting or non adapting after a 5 cGy priming dose in biological replicate experiments. Applicants study design utilized oligonucleotide microarrays containing ˜12,000 human genes. The RNA sampling time (i.e., four hours after the challenge dose) was selected to allow the comparison of new gene-transcript findings with literature reports of corresponding protein changes that may occur within the same time window after the challenge dose.

There are numerous applications for the present invention. For example, the present invention can be used for estimation of IR dose of exposure, for estimation of individual susceptibility to ionizing radiation, for identification of novel IR induced cancer markers, and for identification of IR markers for radiotherapy planning and success evaluation. The panels of genes can be used for the development of biomarkers of radiation exposure and biomarkers of individual susceptibility, which have applications in radiotherapy dose planning to improve treatment cure, and for radiation biosensors for military, civilian, and occupational exposures to ionizing radiation. The present invention can also be used for detectors for clinical exposure to ionizing radiation and for radiation biodosimetry after an accidental, military, or civilian exposure to external beam exposure to ionizing radiation, medical assessment or triage. Other uses of the present invention include identifying molecular targets for manipulating a cell's sensitivity or resistance to ionizing radiation exposures.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 is a graph illustrating radiating cells to identify tissue sensitivity genes.

FIG. 2 is a graph showing cell lines used to identify the panels of tissue sensitivity genes associated with low-dose priming effects.

FIG. 3 is an interaction model of TP53-related genes associated with radioadaptation and illustrates two panels of tissue sensitivity genes.

### DETAILED DESCRIPTION OF THE INVENTION

Referring to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

The present invention involves the identification and demonstration of a multiplicity of panels of human genes that are associated with differential sensitivity to ionizing radiation in human cells. The present invention provides a method of predicting tissue sensitivity to radiation comprising selecting at least one of a first panel of genes associated with increased chromosomal damage and a second panel of genes associated with reduced chromosomal damage, exposing the tissue to radiation, measuring RNA of the tissue providing measured RNA of the first panel of genes associated with increased chromosomal damage and measured RNA of the second panel of genes associated with reduced chromosomal damage, and predicting sensitivity to radiation using at least one of the measured RNA of the first panel of genes associated with increased chromosomal damage and measured RNA of the second panel of genes associated with reduced chromosomal damage. In one embodiment, the step of selecting comprises selecting a first panel of genes associated with increased chromosomal damage and selecting a second panel of genes associated with reduced chromosomal damage, the step of measuring RNA of the tissue comprises measuring RNA of the tissue providing measured RNA of the first panel of genes associated with increased chromosomal damage and comprises measuring RNA of the tissue providing measured RNA of the second panel of genes associated with reduced chromosomal damage, and the step of predicting sensitivity to radiation comprises using the measured RNA of the first panel of genes associated with increased chromosomal damage and using measured RNA of the second panel of genes associated with reduced chromosomal damage. For example, the step of predicting sensitivity to radiation can comprise comparing the measured RNA of the first panel of genes associated with increased chromosomal damage and measured RNA of the second panel of genes associated with reduced chromosomal damage.

Another embodiment of the present invention provides a method of using tissue for predicting sensitivity to radiation comprising the steps of selecting a first panel of genes associated with increased chromosomal damage, selecting a second panel of genes associated with reduced chromosomal damage, exposing the tissue to a priming dose of radiation, waiting for a time period and exposing the tissue to a challenge dose of radiation, waiting for a time period and measuring RNA of the tissue providing measured RNA of the first panel of genes associated with increased chromosomal damage and providing measured RNA of the second panel of genes associated with reduced chromosomal damage, and predicting sensitivity to radiation by comparing the measured RNA of the first panel of genes associated with increased chromosomal damage and the measured RNA of the second panel of genes associated with reduced chromosomal damage.

The gene panels were developed by investigating the cytogenetic adaptive response of human lymphoblastoid cell lines in order to: (a) determine how an initial dose influences subsequent gene-transcript expression in reproducibly adapting and non-adapting cell lines, and (b) identify gene transcripts that are associated with reductions in the magnitude of chromosomal damage after the challenge dose. The transcript profiles were evaluated using oligonucleotide arrays and RNA obtained 4 hours after the challenge dose. A set of 145 genes (false discovery rate=5%) with transcripts that were affected by the 5 cGy priming dose fell into two categories: (a) a set of common genes that were similarly modulated by the 5 cGy priming dose irrespective of whether the cells subsequently adapted or not and (b) genes with differential transcription in accordance with their adaptive or non-adaptive outcomes. The common radiation response genes showed up-regulation for protein synthesis genes and down-regulation of metabolic and signal transduction genes (>10 fold differences). The genes associated with subsequent adaptive and non-adaptive outcomes involved DNA repair, stress response, cell cycle control and apoptosis. Changes in gene expression in some panels of genes are indicators of radiation exposure while other panels are predictive for the risk of subsequent genomic damage and cellular toxicity.

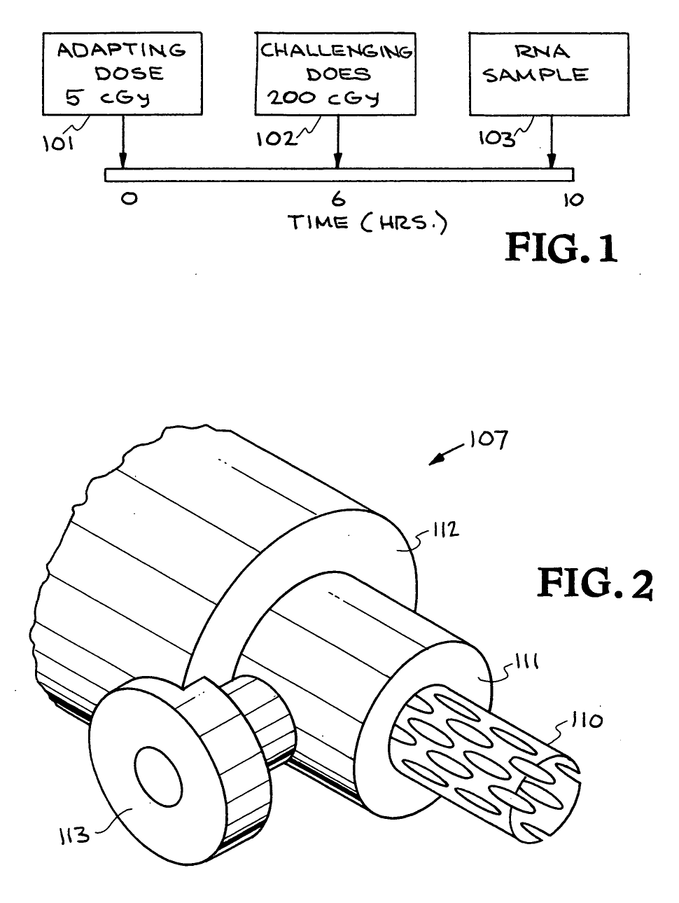

Referring to FIG. 1, one embodiment of the present invention is illustrated by a graph. The graph is designated generally by the reference numeral 100. The graph 100 illustrates a method of predicting tissue sensitivity to radiation. At least two panels of genes are selected that are associated with differential sensitivity to ionizing radiation in the tissue. In the graph 100, an adapting dose of 5 cGy (designated by the reference numeral 101) is administered to the tissue sample as the starting time (Time 0). The next step comprises waiting for a time period. In the graph 100 the waiting time period is 6 hours. In the next step, a challenge dose of 200 cGy (designated by the reference numeral 102) is administered to the tissue sample. The next step comprises waiting for another time period. In the graph 100, the other time period is 4 hours. The next step (designated by the reference numeral 103) comprises measuring RNA of the tissue providing measured RNA of said first panel of genes associated with chromosomal damage and providing measured RNA of said second panel of genes associated with chromosomal damage and predicting sensitivity to radiation by comparing said measured RNA of said first panel of genes associated with chromosomal damage and said measured RNA of said second panel of genes associated with chromosomal damage.

Applicants completed experiments of the present invention. In various examples, tissue sensitivity to radiation was predicted by selecting panels of genes associated with differential sensitivity to ionizing radiation in the tissue, exposing the tissue to a priming dose of radiation, waiting for a time period, exposing the tissue to a challenge dose of radiation, analyzing the tissue, and predicting tissue sensitivity to radiation. Some of the examples are described below. In addition, examples and additional information are described in the October 2005 article “Low-Dose Irradiation Alters the Transcript Profiles of Human Lymphoblastoid Cells Including Genes Associated with Cytogenetic Radioadaptive Response,” by Matthew A. Coleman, Eric Yin, Leif E. Peterson, David Nelson, Karen Sorensen, James D. Tucker, and Andrew J. Wyrobek, in Radiation Research Vol. 164, No. 4, pp. 369-382, October 2005. The October 2005 article “Low-Dose Irradiation Alters the Transcript Profiles of Human Lymphoblastoid Cells Including Genes Associated with Cytogenetic Radioadaptive Response,” by Matthew A. Coleman, Eric Yin, Leif E. Peterson, David Nelson, Karen Sorensen, James D. Tucker, and Andrew J. Wyrobek, in Radiation Research Vol. 164, No. 4, pp. 369-382, October 2005 is incorporated herein by reference.

### EXAMPLE 1

Applicants completed experiments utilizing three human LCLs (GM15036, GM15510 and GM15268) that Applicants had previously characterized for their cytogenetic radioadaptive responses in biological replicate analyses of micronucleus frequencies. Briefly, cells in logarithmic growth in suspension cultures were exposed using a Cesium source to sham or 5 cGy priming dose, followed six hours later by 200 cGy (challenge dose), and then analyzed ˜20 hours later for relative effects of the priming dose versus sham irradiation on the micronuclei frequencies. Aliquots of cells were frozen at multiple times after the challenge dose, and Applicants study focused on samples collected 4 hours after the 200 cGy challenge dose from cultures that had been previously treated with or without the 5 cGy priming dose.

### EXAMPLE 2

Irradiations and RNA preparation: A total of 1×10⁷ cells for each cell line were collected and irradiated using a ¹³⁷Cs Mark 1 Irradiator (J.L. Shepherd and Assoc., Glendale, Calif.) with a priming dose of 5 cGy followed 6 hours later with a challenging dose of 200 cGy. The negative control was sham irradiated (neither priming nor challenge dose) and the positive control received only the challenge dose. Dose rates were 0.3 and 0.6 Gy/min for the priming and challenge doses, respectively. After irradiation, cells were grown for an additional 4 h at 37° C. and then harvested by centrifugation, resuspended in approximately 250 μl of media and frozen at −80° C. Total RNA was extracted using the TRIZOL protocol (Invitrogen). RNA was treated with RNase-free DNase to remove any contaminating genomic DNA (BD Biosciences Clontech, Palo Alto, Calif.), and RNA quality was confirmed by agarose gel electrophoresis with ethidium bromide staining or using an Agilent Bioanalyzer (Agilent Technologies, Palo Alto, Calif.). Purified total RNA was stored at −80° C.

### EXAMPLE 3

Applicants investigated the cytogenetic adaptive response of human lymphoblastoid cell lines exposed to 5 cGy (priming dose) followed by 2 Gy (challenge dose) compared to cells that received a single 2-Gy dose. Applicants investigation was to (a) determine how the priming dose influences subsequent gene transcript expression in reproducibly adapting and non-adapting cell lines, and (b) identify gene transcripts that are associated with reductions in the magnitude of chromosomal damage after the challenge dose. The transcript profiles were evaluated using oligonucleotide arrays and RNA obtained 4 h after the challenge dose. A set of 145 genes (false discovery rate 5.5%) with transcripts that were affected by the 5-cGy priming dose fell into two categories: (a) a set of common genes that were similarly modulated by the 5-cGy priming dose irrespective of whether the cells subsequently adapted or not and (b) genes with differential transcription in accordance with the cell lines that showed either adaptive or non-adaptive outcomes. The common priming dose response genes showed up-regulation for protein synthesis genes and down-regulation of metabolic and signal transduction genes (0.10-fold differences). The genes associated with subsequent adaptive and non-adaptive outcomes involved DNA repair, stress response, cell cycle control and apoptosis. Applicants' findings support the importance of TP53-related functions in the control of the low-dose cytogenetic radioadaptive response and suggest that certain low-dose-induced alterations in cellular functions are predictive for the risk of subsequent genomic damage.

Cell Culture—The LCL samples were obtained from the Coriell Cell Repositories at American Tissue Culture Collection (Manassas, Va.). Use of these publicly available cell lines were deemed exempt from institutional IRB approval. Cells were suspension grown in RPMI 1640 (Invitrogen, Carlsbad, Calif.) supplemented with 15% fetal calf serum (Sigma-Aldrich, Chicago, II) containing antibiotic-antimycotic mixture (100 units/ml penicillin G sodium, 100 μg/ml streptomycin, and 0.25 μg/ml amphotericin B as Fungizone® in 0.85% saline (Invitrogen)), and 2 mM L-glutamine (Invitrogen). All cultures were grown in a humidified 5% CO₂ atmosphere at 37° C. and maintained at a concentration of 1 to 10×10⁵ cells/ml.

Oligonucleotide Microarrays—Isolated RNA was converted to double stranded cDNA following the Expression Analysis Technical Manual (Affymetrix, Santa Clara, Calif.). The Enzo BioArray HighYield Transcript Kit was than used for RNA amplification-labeling (Enzo Biochem, New York, N.Y.). The Affymetrix HU95A gene chips were hybridized using 10 μg of the fragmented complementary RNA followed by processing in an Affymetrix Fluidics Workstation (Affymetrix). Hybridization signals were detected through the use of an argon-ion laser scanner (Agilent Technologies), and output for pixel intensities and confidence calls for each of the genes detected on the array were generated with Affymetrix Microarray Suite 5 (MAS-5) software.

Statistical Analysis—Eighteen Affymetrix HU95A chips were used for this study: two experimental replicate arrays for each of the nine combinations of three cell lines and three treatments. P-values and log₂-transformed intensities were obtained from Affymetrix's MAS-5 software, and normalized in two steps. First, the pair of chips for each replicate was normalized using Astrand's quantile normalization method to produce chips with the same overall intensity distribution. Second, the normalized intensities across the eighteen chips were adjusted by a chip-specific factor to ensure that the median intensity of the 12,626 genes on each chip was identical across the eighteen chips.

MAS-5 p-values were used to determine genes with a positive signal. The p-values for each chip were adjusted by the Benjamini-Hochberg method to control the per-chip False Discovery Rate (FDR). The “mt.rawp2adjp” procedure in Bioconductor was used to perform the adjustment. Only genes with an FDR-adjusted p-value not exceeding 0.01 were selected for subsequent analysis as described below.

The per-chip expression data corresponding to genes with a positive signal were combined in a two-step process to obtain an initial analytic data set consisting of 4,768 genes. The first step consisted of producing three separate cell line data sets. Each cell line data set consisted of expression data for all the genes for which a signal was detected in at least one of the six chips for that cell line. The second step consisted of combining the three cell line data sets into an initial analytic data set. The analytic data set consisted of genes present in any of the three cell line data sets. Thus each gene in the analytic data set could have expression data for one, two, or three cell lines, and might be expressed in one, two or three of the treatment conditions assayed in each cell line.

Genes with differential expression across the nine treatment combinations were detected by means of an F-test. A separate F-test was performed on each gene. Each F-test evaluated differences among three, six, or nine treatments according to whether data were present for one, two, or three cell lines. The p-values for these 4,768 genes were adjusted using the same Benjamini-Hochberg procedure described above. A total of 1,775 genes had an adjusted F-test p-value <0.05, of which there were 1,208 present in all cell lines. These 1,208 genes were selected for further analysis to detect differences in response to radiation exposure across the cell lines. In this context, the “priming dose response” for a cell line is defined to be the difference between the response to a 200 cGy exposure with and without a preceding 5 cGy exposure (5+200 vs 200 cGy). This difference was evaluated using an F-test for interaction between cell line and the two treatments. Only those responses detected in two or more cell lines were examined. The resulting p-values were again adjusted to control for FDR, and 520 genes with an adjusted p-value not exceeding 0.20 were selected for further analysis.

For gene annotation and functional classification Applicants used EASE (the web address for EASE is: www.david.niaid.nih.gov) and MAPPFinder (Gladstone Institute at UCSF, web address for MAPPFinder is (www.genmapp.org); that use the “Gene Ontology” (GO) consortium (the web address for GO is: www.geneontology.org); database for the analysis of pathways and gene relationships. We applied a ratio criterion of greater than 1.8 for (5+200 cGy)/200 cGy signals for the 520 genes using MAPPFinder to search for GO annotations associated with the significant differential transcripts affected by the priming dose. This fold cut-off was based on prior criteria established for microarray data in our laboratory, and is consistent with the literature. The standardized difference scores were used to rank GO categories based on the relative number of gene expression changes within each GO-map. An assigned standardized difference score (z-score;) greater than 2 was used as a measure of effect for both the priming dose and for association with the micronucleus radioadaptive endpoint. The z-score and micronucleus measure of the adaptive response were then used to select candidate genes with ontologies and pathway information.

The analysis tool CLUSter and Factor Analysis Using Varimax Orthogonal Rotation (the web address for CLUSFAVOR is: www.mbcr.bcm.tmc.edu/genepi) was used to identify associations between both priming dose and radioadaptation transcript responses of the 520 genes using their fold change (5+200 cGy/200 cGy). Cluster analysis was based on Euclidean distance without standardization to identify the natural grouping of gene expression profiles. The color gradient was based on percentile of global fold values for all genes and cell lines.

Semi-Quantitative PCR—Confirmation of transcript levels for several associated radioadaptive modulated genes was performed by RT-PCR of cDNA produced from mRNA from each of the three cell lines. RNA from samples used for microarray analyses was reverse transcribed using Superscript II reverse transcriptase (Life Technologies, Rockville, Md.) and an oligo-dT primer. PCR was performed in 100 μl reactions, using the Platinium® RT-PCR Thermoscript One-Step System (Invitrogen, Carlsbad, Calif.). The GAPDH gene, which did not show a change in expression (data not shown), was used as an internal control.

The primers selected were the following:

GAPDH (200 base pair (bp)) Forward-Primer:
TCTAGAAAAACCTGCCAAA,
Reverse-Primer:
TACCAGGAAATGAGCTTGA;
ATM (406 bp) Forward-Primer:
ACCAGAGATATTGTGGATGG,
Reverse-Primer:
TTGAGATTTTTGGGGTCTATG;
P125 Phospholipase (399 bp) Forward-Primer:
TCCAGATTTGGACCTAAAAG,
Reverse-Primer:
CTCTGAAGAGCGAAAAGGTA;
MYC (410 bp) Forward-Primer:
TGAGGAGGAACAAGAAGATG,
Reverse-Primer:
TGAGGAGGAACAAGAAGATG;
IFNR2 (380 bp) Forward-Primer:
CAGTTGGAACTCTTGAGTGG,
Reverse Primer
ATATAACCATCCCCAAGGTC;
HSP8A (404 bp) Forward-Primer:
GGAAGACATTGAACGTATGG,
Reverse Primer
AATCAACCTCTTCAATGGTG;
and
CBF2 (403 bp) Forward-Primer:
GCTCTGGAAAGGATGATATG,
Reverse Primer
GATCCCATATTTTCATCCAA.

PCR conditions were optimized to be performed as follows for all transcripts: 25-30 cycles at 94° C. for 15 seconds; 52° C. for 30 seconds; 72° C. for 1 minute, followed by 1 cycle at 72° C. for 10 minutes.

Referring now to FIG. 2, a graph shows cell lines used to identify the panels of tissue sensitivity genes associated with low-dose priming effects. The graph is designated generally by the reference numeral 200. The graph 200 illustrates Applicants' investigation of gene-transcript profiles of three human LCLs that were previously characterized for their cytogenetic adaptive response (AR) by micronucleus analysis. The three human LCLs are: GM15036 designated by the reference numeral 201, GM1551 designated by the reference numeral 202, and GM15268 designated by the reference numeral 203. Micronucleus analysis was used to quantify the radioadaptive response for three cell lines. The k value is the ratio of micronucleus frequencies in cells that received the priming dose as well as the challenge dose (5+200 cGy) versus cells that received only the challenge dose (200 cGy). The k values of less than one indicate that adaptation occurred, while values equal to or close to one show a lack of adaptation. The white and black bars represent the results of the first and second independent biological replicate experiments, respectively. Cell lines GM15268 and GM1551 were reproducibly adaptive, showing 20-30% reductions in the frequency of micronuclei in cells that received the 5 cGy priming dose followed by a 200 cGy challenge dose. In contrast, line GM15036 was reproducibly non-adapted in replicate experiments, showing no detectable change in micronucleus frequencies associated with the 5 cGy priming dose. RNA was isolated for gene-transcript analyses from three experimental groups for each cell line: 4 h after sham irradiation (0 cGy), 4 h after a dose of 200 cGy that was preceded by a sham priming dose (200 cGy), and 4 h after a challenge dose of 200 cGy that was preceded by a 5 cGy priming dose (5+200 cGy).

Statistical analyses identified a set of genes whose gene-transcript levels were differentially modulated in cells after they had received the 5 cGy priming dose followed by the challenge dose versus cells that received only the 200 cGy challenge dose. The hybridization signals across ˜12,000 oligonucleotide probe sets, i.e., genes, showed little variability between replicate chips (correlation coefficient >0.98). There was detectable signal in at least one experimental group across the three cell lines for 4,768 genes (FDR 1%). Differential gene expression among the three groups identified 1,775 genes (F-ratio, FDR 5%) of which 1,208 genes had detectable gene-transcript signals in all three cell lines for both the 200 cGy and 5+200 cGy samples. A subset of 520 genes showed differential responses (>1.8-fold) between the 200 cGy and 5+200 cGy groups in at least one cell line (FDR 20%; 145 genes with FDR 5%; see supplemental data at microarray.llnl.gov for a complete listing of genes). The 520 genes fell into 713 Gene Ontology (GO) categories with at least one gene per category (see supplemental data at microarray.llnl.gov). The top five GO categories accounted for 40% of the genes: protein biosynthesis (−10%, 54 genes), DNA dependent transcription (8%, 43 genes), ATP binding activity (−8%, 41 genes), immune response (−7%, 38 genes) and regulation of transcription (−7%, 35 genes). Reducing the size of the input list to 145 genes by lowering the FDR to 5% did not significantly alter the distribution of the top GO categories (data not shown).

TABLE 1
Genes that are Up-Regulated in Response to the
Priming Dose, Independent of the Adaptive
Response Outcome (Group 1)^{a}

| | Accession | Relative expression | | | |
|---|---|---|---|---|---|
| Gene | number | GM15036 | GM15510 | GM15268 | Average^{b} |
| EEF1A1 | J04617 | >10 | >10 | 10 | 10.0 |
| RPS2 | X17206 | >10 | >10 | 9 | 9.7 |
| RPL28 | U14969 | >10 | 3.4 | 4.8 | 6.1 |
| RPS15A | W52024 | 10 | 1.7 | 4.7 | 5.5 |
| RPL8 | Z28407 | 7.4 | 2.3 | 6.5 | 5.4 |
| RB1 | NM_000321 | 3.4 | 2.6 | >10 | 5.3 |
| RPLP1 | M17886 | 8.3 | 3.2 | 4.3 | 5.3 |
| RPS4X | M58458 | 9.7 | 1.8 | 4.1 | 5.2 |
| DRAP1 | U41843 | >10 | 3.3 | 2.1 | 5.1 |
| RPS11 | X06617 | 9 | 2.7 | 3 | 4.9 |
| RPS10 | U14972 | 7.1 | 2.8 | 4.2 | 4.7 |
| RPL6 | X69391 | 7.6 | 2.6 | 3.5 | 4.6 |
| RPS20 | L06498 | 8 | 2 | 3.5 | 4.5 |
| GNB2L1 | M24194 | 9.2 | 1.7 | 2.5 | 4.5 |
| RPS3 | X55715 | 6.1 | 3 | 4.1 | 4.4 |
| RPL18A | X80822 | 6.9 | 3.8 | 2.5 | 4.4 |
| RPL10 | M64241 | 5.6 | 3.2 | 4.3 | 4.4 |
| RPL14 | D87735 | 8.3 | 2.5 | 2.2 | 4.3 |
| RPS8 | X67247 | 6.7 | 2.5 | 3.4 | 4.2 |
| RPLP0 | M17885 | 4.7 | 2.8 | 5.1 | 4.2 |

TABLE 2
Genes that are Down-regulated in Response to the
Priming Dose, Independent of the Adaptive
Response outcome (Group 2)^{a}

| | Accession | Relative expression | | | |
|---|---|---|---|---|---|
| Gene | number | GM15036 | GM15510 | GM15268 | Average^{b} |
| SPTLC1 | Y08685 | 0.1 | 0.2 | 0.1 | 0.1 |
| ATP2B1 | J04027 | 0.1 | 0.2 | 0.1 | 0.1 |
| KLAA0004 | D13629 | 0.1 | 0.1 | 0.3 | 0.2 |
| H-SP1 | X68194 | 0.1 | 0.2 | 0.2 | 0.2 |
| GLDC | D90239 | 0.1 | 0.3 | 0.1 | 0.2 |
| TNFRSF8 | M83554 | 0.2 | 0.3 | 0.1 | 0.2 |
| ZMPSTE24 | Y13834 | 0.1 | 0.2 | 0.3 | 0.2 |
| W28612 | W28612 | 0.3 | 0.1 | 0.2 | 0.2 |
| TFRC | X01060 | 0.3 | 0.1 | 0.2 | 0.2 |
| TFRC | M11507 | 0.3 | 0.1 | 0.2 | 0.2 |
| RZF | AF037204 | 0.3 | 0.1 | 0.2 | 0.2 |
| LAMP2 | U36336 | 0 | 0.2 | 0.4 | 0.2 |
| CD19 | M28170 | 0 | 0.2 | 0.4 | 0.2 |
| SLC9A6 | AF030409 | 0.2 | 0.3 | 0.2 | 0.2 |
| SLC7A5 | M80244 | 0.5 | 0.1 | 0.1 | 0.2 |
| HMGCR | M11058 | 0.2 | 0.2 | 0.3 | 0.2 |
| SQLE | D78130 | 0.4 | 0.2 | 0.1 | 0.2 |
| ITGB1 | X07979 | 0.1 | 0.2 | 0.4 | 0.2 |
| TMP21 | L40397 | 0.3 | 0.2 | 0.3 | 0.3 |
| SLC2A5 | M55531 | 0.3 | 0.2 | 0.3 | 0.3 |

The GO categories with differential z-scores and cluster analyses identified four groups of genes whose transcription was modulated by the 5 cGy priming dose. Genes of groups 1 and 2 (Tables 1 and 2) were modulated in the same direction among all cell lines (up or down, respectively), when the challenge dose was preceded by the priming dose, and the responses were independent of their subsequent AR outcomes. Applicants consider these transcripts to be a measure of 5 cGy priming dose and may serve as bioindicators of low-dose exposure. Grouping by functional category for the priming dose genes are shown in Table 3; columns one and two. Group 3 genes (Table 4) were also modulated by the priming dose, but showed higher expression levels after the priming dose in the non-adapted cell line than in the adapted cells lines. Group 4 genes (Table 5) were also modulated by the priming dose, but showed relatively higher expression in the adapted cell lines than in the non-adapted cell line. The Group 3 and 4 transcripts were therefore indicative of radioadaptation. Grouping by functional category for the radioadaptive genes are shown in Table 3; columns three and four.

TABLE 3
Pathway Analysis of Genes Associated with Priming Dose or Radioadaptive Effects

| | Priming dose genes | | Radioadaptive genes | |
|---|---|---|---|---|
| | Group 1, common “up” | Group 2, common “down” | Group 3, differential | Group 4, differential |
| Pathway | response after | response after | response, lower in | response, higher in |
| Response group | priming dose | priming dose | adapting cell lines | adapting cell lines |
| Apop [symbol] osis | | CD53, PORIMIN, | CASP8, DAD1, HAX1, | |
| | | TNFRSF10B | NFKB1, TNF, | |
| | | | TNFRSF17 | |
| Cell adhesion | | CD58, ENTPD1, ITGB1, | CD44, CD164 | ICAM2 |
| | | KIAA0911 | | |
| Cell cycle | RBI | WEE1 | CCNI, BTG1, BUB3, | CEIN3, |
| | | | EDFI, EMP3, MYC | MPHOSPH10, P125 |
| | | | PIM3, SRF, | |
| | | | PRKDCIP | |
| Chemokine | | | CCL3, CCL4, SCYA5, | CRMI |
| | | | SCYA33, SCYA3, | |
| | | | SCYA4 | |
| DNA repair | | PRKDC | PRKARIA | ATM, ERCC5, SP100, |
| | | | | NBP |
| Immune response | | | CD48, IFITMI, HLA- | BLNK, NKTR |
| | | | DMA, HLA-DMB, | |
| | | | TCRA | |
| Metabolism | ODCI, RPSI3 | ARL [symbol] IP, CD9, CYP1B1, | AHCY, ADA, IDH3 | DLAT, FDFTI, LDHB |
| | | EBP, ENTPDI, GGH, | IDH3G, ODC1, | OS9, SUCLA3 |
| | | GLDC, GM [symbol] A, GUSB, | PMM2, SC [symbol] DL, | |
| | | HMGCR, KIAA0004, | SIAT1 | |
| | | KIAA0088, LAMP1, | | |
| | | LAMP2, PPTI, SLC2A5, | | |
| | | SLC9A6, SPTLC1, TFRC, | | |
| | | ZMPSTE24 | | |
| Protein degradation | | KIAA0317, RPN2, RZF, | CISC, EDD, PSENI, | IFT30, PSMA4, |
| | | TLI32, USP9X | SPAG7, UBE21 | PSMC1, PSMO6, |
| | | | | PSMD10, USP6 |
| Protein biosynthesis | RPL (5, 6, 8-12, 13A, 14, | FMR1, CANX, MRPS6 | EIF3SS, EIF4A1, | |
| | 15, 17, 19, 18A, 21, 23, | | E2EPF | |
| | 23A, 24, 27A, 28-32, 34, | | | |
| | 38), RPLP0, RPLP1, RPS | | | |
| | (2, 3, 3A, 4X, 5-11, 14, | | | |
| | 15A, 17, 19, 20, 23, 24, | | | |
| | 27). EEF1AI, EEF1G | | | |
| RNA metabolism | | DDX3, HNRPH3, RNP34 | DDX21, NXFI, | DDX42, RTCD1 |
| | | | PABPCI, SFRS2, | PABPCI, RTCD1 |
| | | | SFRS6 | |
| Signal transduction | DRES9, GNB2LI, TEBP | ADAM10, AKAP1, ATP1B3, | AMFR, BZRP, GNB1, | CREM, CINNBI, |
| | | ASAH, CDI9, CD59, | IFNAR2, NUDT3, | EBNA1, BP2, |
| | | CNIH, CR2, EBI2, EPB72, | PRKCB1, RGS1, | LPXN, PFTKI, |
| | | SLAM, SORLI, TNFRSF8 | STAT1, STAT3, | YWHAQ, ITK |
| | | | SSRP1, STK [symbol] 4, | |
| | | | VEGFB, YWHAZ | |
| Stress response | HADH3 | HSPA5, PDLA3, PDLA6, | HERPUDI, GADD45A, | DIAI, HSPAB, |
| | | PRDX4, SQLE | MIRP, LITAF, | HSPD1, IF130 |
| | | | SERP1, TXNRD1 | |
| Transcription | DRAP1, NSEPI | H-SP1, TRA1, CHD1 | ATF5, EIF4A2, | CBF2, DEK, SMAR- |
| | | | ETR101, JUND, | CA2, TCF12, |
| | | | SMARCA4, T CEAL | ZNF148 |
| Translation | | | GARS, SARS, WARS, | EIF251, MIIF2, |
| | | | YARS | NARS, RARS |

Some genes are associated with priming-dose effects, but independent of AR outcome. The top 12 GO maps with consistently elevated z-scores (>2) across all three cell lines, which identifies cellular pathways involved in the common transcriptional responses across the three cell lines, independent of their adaptive outcomes. Cluster analyses identified two clusters of genes that were similarly modulated by the priming dose across all three cell lines, independently of their AR outcomes: “all up” and “all down.” There was a very large (˜100 fold) range of responses between groups 1 and 2 (Tables 1 and 2, and supplemental data at microarray.llnl.gov). The top 20 genes of Group 1 showed 4-10 fold higher gene-transcript levels in cells that received the priming dose prior to the challenge dose, when compared to cells that received the challenge dose alone (Table 1). The top 20 genes of Group 2 (Table 2) showed 5-10 fold reductions in gene-transcript levels.

TABLE 4
Genes with Relatively Larger Changes
in Expression when the Cell does not Adapt
(Group 3)

| | Accession | Relative expression | | | Ratio of |
|---|---|---|---|---|---|
| Gene | number | GM15036 | GM15510 | GM15268 | effect^{a} |
| SCYA4 | J04130 | 3.3 | 0.03 | 0.03 | 110.0 |
| SCYA3 | D90144 | 2.5 | 0.2 | 0.1 | 16.7 |
| ATF5 | AB021663 | 7.8 | 0.7 | 0.6 | 12.0 |
| IFNAR2 | L42243 | 2.7 | 0.3 | 0.2 | 10.8 |
| JUND | X56681 | 3.2 | 0.3 | 0.4 | 9.1 |
| SFRS6 | AL031681 | 3.2 | 0.3 | 0.4 | 9.1 |
| SARS | X91257 | 8.2 | 1 | 0.8 | 9.1 |
| ETR101 | M62831 | 3.7 | 0.6 | 0.4 | 7.4 |
| LITAF | AF010312 | 6.5 | 1.2 | 0.6 | 7.2 |
| EIF4A1 | D13748 | 4.8 | 1.1 | 0.6 | 5.6 |
| GARS | U09510 | 3.1 | 0.7 | 0.4 | 5.6 |
| MYC | V00568 | 3.9 | 0.5 | 0.9 | 5.6 |
| TM9SF2 | U81006 | 0.8 | 0.2 | 0.1 | 5.3 |
| CD48 | M37766 | 2.1 | 0.4 | 0.4 | 5.3 |
| OAS1 | X04371 | 2.1 | 0.4 | 0.4 | 5.3 |
| ADFP | X97324 | 3.4 | 1 | 0.3 | 5.2 |
| PIM2 | U77735 | 3.6 | 0.9 | 0.5 | 5.1 |
| NP | X00737 | 6.7 | 1.5 | 1.2 | 5.0 |
| PRKDC1P | U85611 | 5.8 | 1.4 | 1 | 4.8 |
| WARS | X59892 | 2.9 | 0.8 | 0.4 | 4.8 |

TABLE 5
Genes Associated with the Adaptive Outcome with Higher Expression in the
Adapting Cell Lines (Group 4)

| | Accession | Relative expression | | | |
|---|---|---|---|---|---|
| Gene | number | GM15036 | GM15510 | GM15268 | Ratio of effects^{a} |
| NKTR | NM_005385 | 0.2 | 6.9 | 4.3 | 28.0 |
| PSMC6 | D78275 | 0.2 | 1.9 | 1.2 | 7.8 |
| PFTK1 | AB020641 | 0.2 | 1.5 | 1.3 | 7.0 |
| MPHOSPH10 | X98494 | 0.5 | 3.2 | 2.5 | 5.7 |
| MTIF2 | AF494407 | 0.3 | 1.5 | 1.6 | 5.2 |
| CRM1 | Y08614 | 0.2 | 1.1 | 0.9 | 5.0 |
| RDX | L02320 | 0.2 | 0.9 | 1.1 | 5.0 |
| TCF12 | M80627 | 0.3 | 1.2 | 1.8 | 5.0 |
| CETN3 | AI056696 | 0.5 | 2.1 | 2.6 | 4.7 |
| FLJ20720 | FLJ20719 | 1.1 | 5.9 | 4.1 | 4.5 |
| PSMA6 | X59417 | 0.3 | 1.7 | 0.9 | 4.3 |
| ZNF148 | AJ236885 | 0.3 | 1.8 | 0.8 | 4.3 |
| C2F | U72514 | 0.5 | 2.8 | 1.5 | 4.3 |
| ERCC5 | L20046 | 0.5 | 2.8 | 1.5 | 4.3 |
| SMARCA2 | X72889 | 0.2 | 1 | 0.7 | 4.3 |
| DDX42 | AB036090 | 0.4 | 1.4 | 1.6 | 3.8 |
| HSPA8 | P11142 | 0.8 | 3.1 | 2.7 | 3.6 |
| EIF2S1 | BC002513 | 0.5 | 1.8 | 1.7 | 3.5 |
| EBNA1BP2 | U86602 | 0.6 | 2.3 | 1.8 | 3.4 |
| DKFZP564F0522 | AK027432 | 0.5 | 1.9 | 1.5 | 3.4 |

Protein synthesis was the major cellular function associated with the common up-regulated genes of Group 1 (Table 3). Strikingly, 48 of 60 Group 1 genes involve ribosomal functions and protein biosynthesis. These genes included the elongation factor EEF1A1 (˜11 fold elevation), which is involved in joining aminoacyl-tRNAs to the ribosomes and various structural protein components of ribosomes (e.g., RPL28, RPS2 and RPS15A). Several cell-cycle and signal-transduction genes were also identified as up-regulated, including GNB2L1 (commonly known as RACK) and the tumor suppressor gene, RB1.

In contrast, the common down-regulated Group 2 genes (Table 3) were dominated by metabolism functions (e.g., GLDC, LAMP2 and KIAA0004). Other Group 2 genes encoded multiple membrane-bound proteins such as ion transporters and proteins involved in cell adhesion related functions (e.g., ATP2B1, CD19, CD44, CD53 and the transferring receptor TFRC and the sodium-hydrogen exchanger SLC96A). Two Group 2 genes were directly associated with cell cycle control and DNA repair pathways (WEE1 and PRKAR1A/protein kinase, cAMP-dependent).

Some gene transcripts are associated with differential AR outcomes (adaptive and non-adaptive). The 520-gene set was also analyzed to identify priming-dose affected genes whose transcript levels were differentially associated with either adaptive or non-adaptive outcomes. The hybridization signals for the two cell lines that reproducibly radioadapted (GM15510 and GM15268) were more highly correlated with each other (correlation coefficient=0.75) than with the non-adapted cell line (GM15036) (0.55 and 0.56 respectively), suggesting that there were global transcriptional changes associated with two possible outcomes; adaptive or non-adaptive.

The GO categories and hierarchical clustering identified two groups of genes associated with AR outcomes (Group 3 in Tables 4 and Group 4 in Table 5, also see supplemental material). Group 3 represented genes with higher transcript levels in the non-adapted cell line and with no detectable change or down-regulated expression in the two adapted cell lines. Group 4 represented genes with higher transcript levels in adapted cell lines and with down-regulated or non-detectable expression changes in the non-adapted cell line. Tables 4 and 5 list the top 20 genes in groups 3 and 4, respectively, and illustrate the large (>100 fold) range of responses between responses in these two groups.

The genes of groups 3 and 4 represented diverse cellular functions associated with AR outcomes (Table 3). Group 3 (i.e., genes with lower transcript levels in adapting cell lines) includes genes associated with cell cycle/proliferation and signal transduction (e.g., MYC, STAT1, BTG1, CCNI, and GNB1); apoptosis-related genes (e.g., TNF, CASP8, and NFKB1); ubiquitin-dependent protein degradation genes (e.g., E2EPF, EDD, KIAA0317, PSMA6, UBE21, USP6, and USP9X); translational control genes involving amino acid activation and tRNA ligation (e.g., GARS, WARS, and YARS); protein modification genes involved specifically in stress response (PRDX4 and GADD45A); DNA double strand break repair (i.e., PRKDCIP;XRCC7); and oxidoreductases genes related to general cellular stress responses (e.g., TXNRD1, IDH2, MTRR, PDIA3 and PDIA6).

Group 4 (i.e., genes with higher expression in adapting cell lines) includes genes involved in DNA-repair (e.g., ATM, SP100, and ERCC5/XPG); cell cycle control and signal transduction (e.g., CETN3, MPHOSPH10, P125 and CREM, EBNA1BP2 and LPXN); and stress response (e.g., PRDX1, HSPA8/HSP70 and HSPD1/HSP60). Group 4 also included six functionally uncharacterized and/or non-annotated genes (i.e., FRG1, RES4-25, DKFZP564, F0522, MEP50, NME1, and CBF2).

AR-associated genes that were linked to TP53 functions—Several Group 3 and 4 genes that discriminated between adaptive and non-adaptive outcomes encode proteins that have been associated with TP53-relted functions. The cell lines that had a adaptive response up-regulated a groups of genes associated with DNA repair and stress response, while down regulating genes associated with cell cycle control and apoptosis, when contrasted to the results for the line that did not adapt after the priming-challenge dosing regimen. The microarray findings for key genes were validated by RT-PCR based on their position on the left and right side of the AR map, for example DNA repair and stress responses (ATM) and Cellular proliferation (MYC). DNA damage sensing was implicated by increased transcript levels in adapted lines for phosphatidyl inositol kinases (i.e., ATM and YWHAQ/14-3-3 family of proteins) which are known to be involved in multiple signaling interactions with mitogen activated kinases such as MAPKp38 and CHK2, which are themselves capable of forming protein-protein interactions with TP53. There were >2 fold increases in the expression levels of ATM in both adapted lines, with no change in the non-adapted line, as confirmed by RT-PCR. In addition, both P125 a cell cycle related transcript and CBF2 a CAAT transcription factor, showed a >2 fold increase on the microarray, and the differential expression was confirmed by RT-PCR. The involvement of nuclear foci in adaptation was implicated by transcript increases in the SP100 gene that interacts with PML, a possible regulator of TP53 function. DNA repair was implicated by increases in ERCC5 transcripts in the lines that adapted. HSP70 family members (HSPA8 (HSP70) and HSPD1 (HSP60)) were also up-regulated in the cell lines that adapted, which can be related to a role in downstream protein degradation. The HSPA8 microarray transcript findings were confirmed by RT-PCR.

Several genes with lower expression in the adapted lines suggested another set of TP53 functional links to cellular proliferation and apoptosis (e.g., CASP8, JUND, MYC, NFKB, SSRP1, TNF, and IFNAR2). The MYC and IFNAR2 transcript microarray finding was verified by RT-PCR. Interestingly, these transcripts showed increased levels in the non-adapted cell line when compared to the adapted lines, suggesting a role for apoptotic gene modulation in AR outcomes. Transcript levels for several transcription factors controlled by NFKB induction were also lower in the radioadaptive cell lines, such as STAT1 and STAT3 that control cell proliferation. The GADD45A stress response gene was up-regulated in the non-adapted line, suggesting differential DNA damage responses for adaptive and non-adaptive outcomes.

### EXAMPLE 4

In one embodiment, the present invention is a method of using tissue for predicting sensitivity to radiation. In the method a first panel of genes associated with increased chromosomal damage is selected. The panel of genes associated with increased chromosomal damage comprises at least one of the following genes: SCYA4, SCYA3, ATF5, IFNAR2, JUND, SFRS6, SARS, ETR101, LITAF, EIF4A1, GARS, MYC, TM9SF2, CD48, OAS1, ADFP, PIM2, NP, PRKDC1P, and WARS. The genes associated with increased chromosomal damage are described in greater detail in Table 4.

In the method a second panel of genes associated with reduced chromosomal damage is selected. The panel of genes associated with reduced chromosomal damage comprises at least one of the following genes: NKTR, PSMC6, PFTK1, MPHOSPH10, MTIF2, CRM1, RDX, TCF12, CETN3, FLJ20720, PSMA6, ZNF148, C2F, ERCC5, SMARCA2, DDX42, HSPA8, EBNA1BP2, AND DKFZP564FO522. The genes associated with DECREASED chromosomal damage are described in greater detail in Table 4.

In the method the tissue is exposed to a priming dose of radiation. The priming dose of radiation is a low dose of radiation less than 1 Gy. The next step comprises waiting for a time period and exposing the tissue to a challenge dose of radiation. For example the next step can be waiting for at least six hours and exposing the tissue to a challenge dose of radiation. The challenge dose of radiation comprises more than 1 Gy but less than 5 Gy.

The next step comprises waiting for a time period and measuring RNA of the tissue providing measured RNA of the first panel of genes associated with increased chromosomal damage and providing measured RNA of the second panel of genes associated with reduced chromosomal damage. For example, the next step can be waiting for at least six hours and measuring RNA of the tissue.

The next step comprises predicting sensitivity to radiation by comparing the measured RNA of the first panel of genes associated with increased chromosomal damage and the measured RNA of the second panel of genes associated with reduced chromosomal damage.

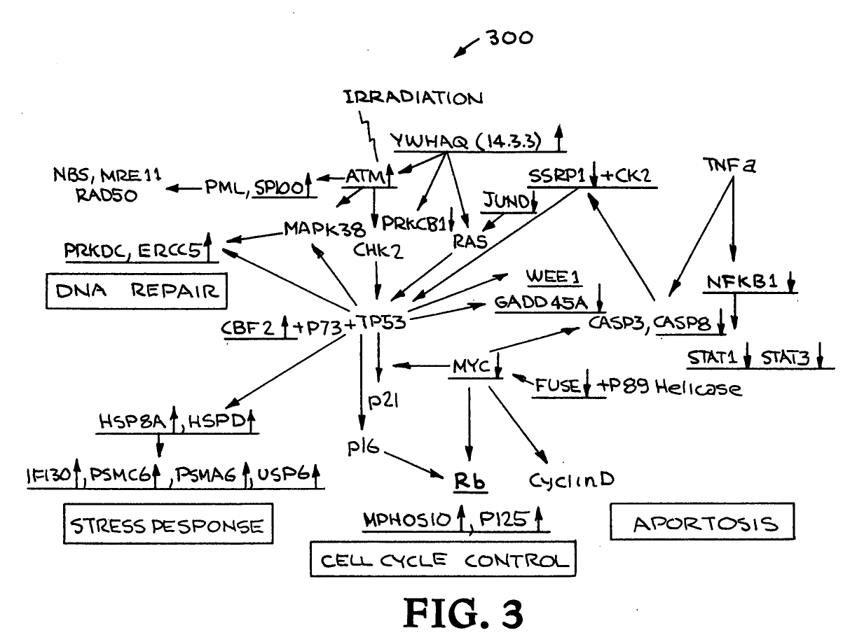

Referring now to FIG. 3, an interaction model of TP53-related genes associated with radioadaptation is illustrated. Based on transcript analysis, this model shows putative interactions between various genes involved in DNA repair, stress response, cell cycle control and apoptosis. The proteins encoded by these genes are directly or indirectly associated with TP53 functions. Underlined genes showed transcript changes in our study. Genes with up arrows had higher transcript levels when cells adapted, and those with down arrows had lower transcripts when cells adapted. Proteins shown in black illustrate linkages based on prior literature. Genes that are only underlined are TP53 related but showed a common 5-cGy priming dose effect with no evidence of differential transcription between adapted and non-adapted cell lines. The P125 and MPHOS10 transcripts are for novel cell cycle genes we identified as up-regulated when cells adapt and their relationship to TP53 functions is not yet established. A corollary figure can be drawn for cells with nonadapting responses.

Applicants' gene-transcript study demonstrates that exposure of LCLs to low-dose radiation (5 cGy) followed six hours later with a high-dose exposure (200 cGy) altered the transcription profiles of a large numbers of genes as measured four hours after the 200 cGy exposure. The gene-transcripts responses fell into two broad categories, (1) those with common responses across all three cell lines, independent of their AR outcomes, and (2) those with differential responses associated with their AR outcomes. Thus, Applicants' study is a simultaneous investigation of expression changes that persist after low-dose exposure, as well as expression changes that may be predictive of the likelihood of subsequent cytogenetic damage. In contrast, previous studies of the effects of low-dose ionizing radiation on transcript levels have been limited to the effects of single acute exposures or repeated high-dose exposures for measuring outcomes. Applicants' study is also the first genome-wide inspection of transcript profiles associated with AR outcomes.

Table 3 contrasts the cellular functions of genes associated with the common priming dose responses versus those associated with the AR outcomes. There was >100 fold range of responses among the set of common priming dose genes (Tables 1 and 2). The >10-fold up and >10-fold down responses are much larger than the magnitude of the typical changes reported after single acute low-dose exposures which are seldom above 3-fold, suggesting that low-dose IR effects were amplified with time or the challenge-dose exposure. Also, there was a surprisingly large range of responses (>100 fold) among priming-dose induced genes with differential gene-transcript levels associated with AR outcomes (Tables 4 and 5).

The major cellular functions associated with the common low-dose primary effects were protein synthesis, metabolism and signal transduction. As shown in Table 3, the protein synthesis genes were generally up regulated, while metabolism genes were generally down regulated; with signal transduction genes showed mostly down but more mixed response. Applicants' findings of increased transcript levels in a large number of protein synthesis genes are consistent with the suggestions that protein biosynthesis is a fundamental response to low-dose IR. Increased protein synthesis is an important component of stress response mechanisms after exposure to IR or other genotoxic agents. The ribosomal transcripts RPL23A, RPL27A, and RPL28 were previously reported to be elevated in mice after low-dose IR exposures, suggesting that low-dose IR elicits a cellular requirement for de novo protein synthesis. However, Applicants' findings of similar responses across all three cell lines irrespective of their AR outcomes, indicate that protein biosynthesis alone is not a critical regulator of radioadaptation, as was previously suggested.

The group 2 down-regulated metabolism genes code for proteins that are membrane-bound or related to mitochondrial processes (e.g., ATP1B3, CYP1B1, SLC2A5, and SLC9A6; Table 3). In yeast, mitochondrial-related processes are known to be modulated after low-dose IR, and changes in the transcript levels of metabolic enzymes involved in oxidation/reduction reactions have been previously shown to be part of a low-dose mitochondrial stress response. Thus, the general reduction in gene transcripts for metabolic enzymes may be related to an overall decrease in the oxidative capacity of cells that had received a priming dose versus those that did not. Two genes associated with cell cycle control and DNA repair (WEE1 and PRKAR1A/protein kinase, cAMP-dependent) were down-regulated while RB1 was up-regulated, suggesting complex roles for cell cycle control cells that received the priming dose.

Previous to Applicants' study, the pathways that control AR were inferred only from studies of individual transcripts and proteins. Using gene chips with cells lines that adapted or not, Applicants identified 100's of candidate genes with differential transcript expression levels associated with AR outcomes. These genes are diverse in their putative functions (Table 3), involving DNA repair, cell-cycle control, chemokines, apoptosis, as well as transcription and translation. Accordingly, Applicants' data suggest that the regulation of AR may involve groups of genes (rather than individuals) whose regulation is juxtaposed, depending on whether the cells will adapt or not. For DNA repair, stress responses, proliferative and apoptotic pathways. Applicants' findings predict that cells will adapt (i.e., show less chromosomal damage) when DNA repair and stress response genes are up-regulated at the same time that certain apoptosis and cell cycle-control genes are down regulated. Alternatively, cells will not adapt (ie., show no change in the amount of chromosomal damage) when the gene-transcript balance is shifted in the other direction (i.e., DNA repair and stress response genes are down regulated, while apoptosis and cell cycle control genes are up-regulated).

Applicants' microarray results point to a critical role for TP53-related pathways in the control of the AR phenomenon, and are consistent with several prior investigations of individual proteins in these pathways. TP53-related pathways have been implicated in AR by affecting both DNA repair and apoptosis pathways related to functional TP53. Applicants' microarray data suggest the involvement of DNA repair in AR via the up-regulation of ERCC5 and ATM. The ERCC5 protein is involved in transcription-coupled repair of oxidative damage and nucleotide excision repair and has also been associated with AR. ATM connects DSB recognition with modulation of TP53 functions. Prior studies with ATM null mice suggested that ATM is not involved in AR, which may be due to ATR complementing the ATM functions of null mice. Applicants' microarray data suggest that ATM up-regulated transcripts are associated with AR outcomes in LCLs, possibly for enhancing repair in response to IR-induced DNA damage via TP53 phosphorylation for cell-cycle arrest or cell death by apoptosis.

Applicants' microarray data identify two stress-related processes related to AR: chromatin remodeling and heat shock responses, both of which are related to TP53 function. CBF2 was up-regulated in cells that adapted (Table 3) and the CBF2 protein can interact with TP53 and P73 to modulate HMG1 gene expression via changes in chromatin structure. The HSP70 genes that are known to be involved in an IR-inducible stress response mechanism and were up-regulated in cells that adapted (Table 3). Induction of HSP70 genes prior to stress exposures has been reported to suppress TP53 expression, greatly decrease BAX levels, and inhibit apoptosis. In a prior study of HSP70 responses under adaptive conditions, HSPA8 transcript levels were not associated with AR responses in mouse splenocytes. However, this experiment failed to show induction of another HSP70 family member PBP74, that was previously associated with AR, suggests that there may be cell-type and tissue-specific variations in the genes associated with AR. Applicants' microarray data suggest that HSP70 response mechanisms are critical components of the control of AR response in human lymphoblastoid cells.

Applicants' data also implicate TP53-related cell-cycle control and apoptotic functions in the control of AR. Example genes include MYC, JUND, TNF, NFKB, CASP8, STAT1 and STAT3, which generally showed decreased levels in adapting cells compared to non-adapting cells. MYC is an important link in the control of cell-cycle proliferation and apoptosis. It is a principal determinants in the TP53 DNA damage pathway, regulating various interactions such as the transcriptional regulation of both CDKN1A (p21/CIP1). Such interactions could prevent cell cycle arrest, which may be needed for efficient DNA repair processes. Others have also observed a down regulation of the MYC transcript after IR, which is consistent with the suggestion that cells normally down regulate MYC to enhance cell survival in response to genotoxic stress. Alternatively, induced levels of the MYC protein and transcript may lead to genomic instability and/or apoptosis. Applicants' observed transcript changes for TNF may also implicate cellular apoptosis in the AR phenomenon. The TNF protein is associated with activation of NFKB, CASP8, STAT1 and STAT3, all of which can affect entry into the cell cycle and apoptosis. Down regulating TNF protein and other small cytokines may be important for maintaining cell-cycle arrest, which is also likely to be important for efficient DNA repair.

Applicants' study design has several notable strengths. The large number of genes assayed enabled the discovery of new genes, new groupings of genes, and complex patterns of transcription in response to IR. The transcription analyses were performed on cells obtained from within the same experiments assayed for micronucleus frequencies to assess radioadaptive capacity of the cells; this nested design was critical because cell lines do not consistently show adaptation. Applicants' approach to normalizing arrays and selecting subsets of potential genes for further evaluation is based on statistical methods developed to analyze and filter data from large expression arrays in a realistic and understandable way. Such an approach allowed Applicants to rank the genes in order of interest using techniques with known, predictable properties and behavior. The comparative tools EASE and GO provided insight into the underlying pathways and functions associated with the common priming dose effects and with the AR outcomes.

Applicants' study design has several limitations that will require further study. Applicants used micronucleus frequency as the measure of AR outcome, and it remains to be determined whether Applicants' findings will be applicable to other measures of adaptation, such as cell survival and genomic instability. A small number of cell lines were studied and only one time point was evaluated. Further research is needed to determine how generalizable the results will be to whole organisms and other cell types. Specifically, epithelial cells tend to undergo growth arrest after IR exposure, whereas lymphoid cells tend to undergo p53-dependent apoptosis. Also, the use of GO categories was problematic because of the multiplicity of functions that can be assigned to any one protein, making it difficult to ascribe a single pathway or function to a gene. For example, TNF and MYC were identified within multiple maps such as Hs_cell, Hs_response to wounding 2, Hs_response to viruses, and Hs_response to biotic stimulus 5, as well as others (Table 3). Also, it remains essentially unknown how transcript changes (up or down) for genes are related to changes in protein levels, protein modifications, or cell fate. For some genes we already have evidence of protein changes associated with transcript changes. For example, transcript findings for HSPA8 and HSPD1 may be associated with changes in protein levels, since HSP70 transcription has been shown to correlate with increased HSP70 protein levels after IR exposures.

The findings support Applicants' three hypotheses. First, exposures to 5 cGy priming doses lead to changes in transcription that persisted beyond the much larger challenge dose. Two broad categories of primer-dose responsive genes were found: (1) genes with common responses after the priming dose, independent of whether the cell lines showed a cytogenetic adaptive response or not (the major effects were up-regulation of genes associated with protein synthesis and down-regulation of metabolism genes) and (2) genes whose transcript were differentially expressed in accordance with whether the cells subsequently adapt or not (the AR response appeared to be associated with differential expression of diverse genes, and we proposed that it is controlled in part by a balance between two sets of TP53 linked pathways: DNA repair/stress response genes versus cellular proliferation/apoptotic genes). Applicants' study findings also generate new hypotheses. Further work will be needed to determine whether low-dose induced transcript alterations are associated with protein changes and whether controlling the expression of genes in the underlying pathways will correspondingly alter survival and residual genomic damage. Further studies will also be needed to determine whether the same pathways regulate low-dose induced AR in tumor cells in vitro and in vivo. This may lead to new insights and technologies for managing and controlling the consequences of exposure to ionizing radiation in radiotherapy, from occupational exposures, and after unexpected radiation exposure incidents.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is:

1. A method of predicting tissue sensitivity to radiation, comprising the steps of:
selecting at least one of a first panel of genes associated with increased chromosomal damage and a second panel of genes associated with reduced chromosomal damage,
exposing the tissue to radiation,
measuring RNA of the tissue providing measured RNA of said first panel of genes associated with increased chromosomal damage and measured RNA of said second panel of genes associated with reduced chromosomal damage, and
predicting sensitivity to radiation using at least one of said measured RNA of said first panel of genes associated with increased chromosomal damage and measured RNA of said second panel of genes associated with reduced chromosomal damage.

2. The method of claim 1 wherein
said step of selecting comprises selecting a first panel of genes associated with increased chromosomal damage,
said step of measuring RNA of the tissue comprises measuring RNA of the tissue providing measured RNA of said first panel of genes associated with increased chromosomal damage, and
said step of predicting sensitivity to radiation comprises using said measured RNA of said first panel of genes associated with increased chromosomal damage.

3. The method of claim 1 wherein
said step of selecting comprises selecting a second panel of genes associated with reduced chromosomal damage,
said step of measuring RNA of the tissue comprises measuring RNA of the tissue providing measured RNA of said second panel of genes associated with reduced chromosomal damage, and
said step of predicting sensitivity to radiation comprises using said measured RNA of said second panel of genes associated with reduced chromosomal damage.

4. The method of claim 1 wherein
said step of selecting comprises selecting a first panel of genes associated with increased chromosomal damage and selecting a second panel of genes associated with reduced chromosomal damage,
said step of measuring RNA of the tissue comprises measuring RNA of the tissue providing measured RNA of said first panel of genes associated with increased chromosomal damage and comprises measuring RNA of the tissue providing measured RNA of said second panel of genes associated with reduced chromosomal damage, and
said step of predicting sensitivity to radiation comprises using said measured RNA of said first panel of genes associated with increased chromosomal damage and using measured RNA of said second panel of genes associated with reduced chromosomal damage.

5. The method of claim 1 wherein said step of predicting sensitivity to radiation comprises comparing said measured RNA of said first panel of genes associated with increased chromosomal damage and measured RNA of said second panel of genes associated with reduced chromosomal damage.

6. The method of claim 1 wherein said step of exposing the tissue to radiation comprises exposing the tissue to a priming dose of radiation, waiting for a time period, and exposing the tissue to a challenge dose of radiation.

7. The method of claim 1 wherein said steps of exposing the tissue to radiation and measuring RNA of the tissue comprise exposing the tissue to a priming dose of radiation, waiting for a time period, exposing the tissue to a challenge dose of radiation, waiting for another time period, and measuring RNA of the tissue.

8. A method of predicting tissue sensitivity to radiation, comprising the steps of:
selecting a panel of genes associated with increased chromosomal damage,
selecting a panel of genes associated with reduced chromosomal damage,
exposing the tissue to radiation,
measuring RNA of the tissue, and
predicting sensitivity to radiation using said panel of genes associated with increased chromosomal damage and said panel of genes associated with chromosomal damage.

9. The method of claim 8 wherein said step of measuring RNA of the tissue provides measured RNA of said genes associated with increased chromosomal damage and measured RNA of said genes associated with reduced chromosomal damage and wherein said step of predicting sensitivity to radiation comprises comparing said measured RNA of said genes associated with increased chromosomal damage and said measured RNA of said genes associated with reduced chromosomal damage.

10. The method of claim 9 wherein said step of exposing the tissue to radiation comprises exposing the tissue to a priming dose of radiation, waiting for a time period, and exposing the tissue to a challenge dose of radiation.

11. The method of claim 8 wherein said steps of exposing the tissue to radiation and measuring RNA of the tissue comprise exposing the tissue to a priming dose of radiation, waiting for a time period, exposing the tissue to a challenge dose of radiation, waiting for another time period, and measuring RNA of the tissue.

12. A method of predicting tissue sensitivity to radiation, comprising the steps of:
selecting at least two panels of genes associated with chromosomal damage,
exposing the tissue to a priming dose of radiation,
waiting for a time period,
exposing the tissue to a challenge dose of radiation,
waiting for another time period,
measuring RNA of the tissue, and
predicting sensitivity to radiation using the measured RNA of said step of measuring RNA of said two panels of genes in the tissue.

13. The method of claim 12 wherein said step of exposing the tissue to a priming dose of radiation comprises exposing the tissue to a dose of radiation less than 1 Gy.

14. The method of claim 12 wherein said step of exposing the tissue to a challenge dose of radiation comprises exposing the tissue to a dose of radiation of more than 1 Gy.

15. The method of claim 1 wherein said step of exposing the tissue to a challenge dose of radiation comprises exposing the tissue to a dose of radiation of more than 1 Gy but less than 5 Gy.

16. The method of claim 12 wherein said step of waiting for a time period comprises waiting for more than one hour.

17. The method of claim 12 wherein said step of waiting for another time period comprises waiting for at least one hour.

18. The method of claim 12 wherein said step of predicting tissue sensitivity to radiation utilizes differential adaptive response outcomes of said step of measuring RNA of the tissue.

19. The method of claim 12 wherein said step of predicting tissue sensitivity to radiation utilizes effects of said priming dose of radiation versus effects of said challenge dose of radiation.

20. The method of claim 12 wherein said step of predicting tissue sensitivity to radiation utilizes measured RNA of said at least two panels of genes associated with chromosomal damage.

21. A method of using tissue for predicting sensitivity to radiation, comprising the steps of:
selecting a first panel of genes associated with increased chromosomal damage,
selecting a second panel of genes associated with reduced chromosomal damage,
exposing the tissue to a priming dose of radiation,
waiting for a time period and exposing the tissue to a challenge dose of radiation,
waiting for a time period and measuring RNA of the tissue providing measured RNA of said first panel of genes associated with increased chromosomal damage and providing measured RNA of said second panel of genes associated with reduced chromosomal damage, and
predicting sensitivity to radiation by comparing said measured RNA of said first panel of genes associated with increased chromosomal damage and said measured RNA of said second panel of genes associated with reduced chromosomal damage.

22. The method of using tissue for predicting sensitivity to radiation of claim 21 wherein said step of exposing the tissue to a priming dose of radiation comprises exposing the tissue to a low dose of radiation less than 1 Gy.

23. The method of using tissue for predicting sensitivity to radiation of claim 21 wherein said step of exposing the tissue to a challenge dose of radiation comprises exposing the tissue to a dose of radiation of more than 1 Gy.

24. The method of using tissue for predicting sensitivity to radiation of claim 21 wherein said step of exposing the tissue to a challenge dose of radiation comprises exposing the tissue to a dose of radiation of more than 1 Gy but less than 5 Gy.

25. The method of using tissue for predicting sensitivity to radiation of claim 21 wherein said step of waiting for a time period and exposing the tissue to a challenge dose of radiation comprises waiting for at least six hours and exposing the tissue to a challenge dose of radiation.

26. The method of using tissue for predicting sensitivity to radiation of claim 21 wherein said step of waiting for a time period and measuring RNA of the tissue comprises waiting for at least six hours and measuring RNA of the tissue.

27. The method of using tissue for predicting sensitivity to radiation of claim 21 wherein said step of predicting sensitivity to radiation utilizes differential adaptive response outcomes of said step of measuring RNA of the tissue.

28. The method of using tissue for predicting sensitivity to radiation of claim 21 wherein said step of predicting sensitivity to radiation utilizes effects of said priming dose of radiation versus effects said challenge dose of radiation.

29. The method of using tissue for predicting sensitivity to radiation of claim 21 wherein said step of predicting sensitivity to radiation utilizes radiation responsive gene expression that is predictive of the magnitude of the radiation toxicity to cells, as measured by the amount of chromosomal damage in said cells after radiation exposure.
